(19) United States
(12) Patent Application Publication
(10) Pub. No.: US 2008/0058405 A1
(43) Pub. Date: Mar. 6, 2008

(54) REPLICATING MELATONIN SIGNALING IN A FETUS, NEWBORN OR SUCKLING INFANT

(75) Inventor: Alfred J. Lewy, Portland, OR (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: OREGON HEALTH & SCIENCE UNIVERSITY, Portland, OR (US)

(21) Appl. No.: 11/831,544

(22) Filed: Jul. 31, 2007

Related U.S. Application Data
(60) Provisional application No. 60/820,913, filed on Jul. 31, 2006.

(51) Int. Cl. A61K 31/4045; A61P 43/00

(52) U.S. Cl. 514/415

(57) ABSTRACT

A method for achieving a chronobiologic effect on a circadian rhythm in a human infant or fetus in utero is described. The method involves the administration of melatonin or dose-equivalent melatonin analogue or melatonin receptor agonist to an infant by administering melatonin or dose-equivalent melatonin precursor, melatonin analogue or melatonin receptor agonist to the infant's breast-feeding mother or wet nurse, and to a fetus by administration of melatonin or dose-equivalent melatonin precursor, melatonin analogue or melatonin receptor agonist to the pregnant woman. Alternatives wherein melatonin or dose-equivalent melatonin analogue or melatonin receptor agonist is administered directly to the infant, such as in formula or in expressed breast milk, are also provided.

### BACKGROUND OF THE INVENTION

1. Field of the Invention

This application relates to regulation of circadian rhythms in humans, particularly circadian rhythms related to the sleep/wake cycle, and to synchronization of such circadian rhythms between mother and child and with the external environment. Specifically, this invention describes a method to achieve a chronobiologic effect, i.e., a biological night-signaling, circadian phase-resetting or related biological-rhythm effect, in humans, particularly infants and especially for newborn infants and fetuses in utero. In particular, this invention relates to enhancement of synchronization of the fetus to the mother and to reestablishment of synchrony between an infant and its mother that is severed upon birth, and to synchronize the infant's endogenous biological circadian rhythms to those of its mother and the external environment. The invention particularly provides methods for synchronizing an infant to a desired sleep time accommodating to the mother.

2. Background of the Related Art

The phenomenon of circadian rhythms in biology is well known, and circadian rhythms are exhibited by all eukaryotic plants and animals, including man. Biological rhythms are periodic fluctuations in biological properties over time; these include circadian as well as seasonal variations. Circadian, or approximately 24-hour, rhythms include production of biological molecules such as hormones, regulation of body temperature, and behaviors such as wakefulness, sleep and periods of activity. In nature, circadian rhythms are closely tied to environmental cues that impose a 24-hour pattern on many of these fluctuations. Experimental inquiry has established that when these cues are absent, most circadian rhythms have a periodicity of approximately 24.5 hours. Circadian rhythms that are not regulated by environmental cues are said to be free-running. The regulation of circadian rhythms by signals from the environment is said to involve entrainment of circadian rhythms. The environmental signals that effect entrainment have been termed zeitgebers, the most important of which is the light/dark cycle.

It is thought in the art that control of circadian rhythms in mammals is mediated by a portion of the brain called the suprachiasmatic nuclei (SCN). Circadian rhythms are mediated by a feedback loop involving the retina, the SCN and the pineal gland. The pineal gland is primarily responsible for the production of melatonin, or N-acetyl-5-methoxytryptamine. Melatonin is believed to be the chemical signal of darkness in mammals, its approximately 12-hour duration conveying the “biological night.”

Fetuses in utero, as well as pre-mature, immature and newborn infants, typically experience at least a period characterized by erratic sleep schedules, including sleeping sporadically during the day and night. These characteristics are consistent with either a chaotic or a free-running circadian rhythm of sleep and wakefulness. Infants, particularly newborn infants, do not have an SCN that is sufficiently developed to properly regulate their own circadian rhythms.

Even after the SCN develops, infants do not respond properly to the light/dark cycle zeitgeber until they are about three months old (about 50 weeks post-conception). Thus, circadian rhythms in infants before the age of three months free-run. The most relevant rhythm in this regard is the sleep rhythm, since synchrony with the infant's environment, particularly the wake/sleep patterns of its parents, are important for optimal rest and interactions within the family. These aspects of infant physiology before the age of about three months preclude appropriately-phased effective synchronization of their circadian rhythms using methods more appropriate (and efficacious) for older infants and adults. See, for example, co-owned U.S. Pat. No. 5,716,978 to Lewy and Sack, and U.S. Pat. Nos. 5,549,683 and 5,641,801 to Wurtman; U.S. Pat. No. 6,703,412 to Rosenthal.

The use of melatonin to synchronize wake/sleep patterns in humans older than about three months, particularly when such patterns have been disrupted, has been disclosed in the art. As disclosed in co-owned U.S. Pat. No. 5,716,978, the art taught melatonin administration to infants having free-running circadian rhythms at administration times that were generally several hours prior to the expected dim light melatonin onset times in the infant, in order to produce the greatest phase advance necessary to counteract the daily phase delay that usually occurs in free-running rhythms. In contrast, for fetuses in utero or infants less than about three months of age, optimal melatonin administration times may be the times that the fetus would normally receive melatonin from its mother, that is, beginning with the time of the mother's melatonin onset or slightly earlier, but not several hours earlier as taught in U.S. Pat. No. 5,716,978. Finally, the art did not teach administering melatonin to a fetus in utero via a pregnant woman or to an infant through breast milk via a nursing mother or wet nurse, since the advantages as disclosed herein of these administration routes was not appreciated.

### SUMMARY OF THE INVENTION

This invention provides methods for synchronizing circadian rhythms in a human fetus or infant, particularly a newborn infant, full-term or premature, and especially circadian rhythms regulating the sleep/wake cycle. The invention particularly provides methods for synchronizing an infant's sleep/wake cycle to a time or pattern accommodating to the mother or primary caretaker. In the inventive methods provided herein, exogenous melatonin or dose-equivalent melatonin analogue, melatonin precursor or melatonin receptor agonist is provided to the infant, preferably in breast milk and more preferably in suckled breast milk, at times that result in the infant's sleep/wake cycle occurring at times accommodating and/or in synchrony with, the mother, wet nurse or other primary caretaker. In certain embodiments, an infant's mother (or wet nurse) receives (by oral ingestion or otherwise) an amount of exogenous melatonin or dose-equivalent melatonin analogue, melatonin precursor or melatonin receptor agonist, to provide an amount of said exogenous melatonin or dose-equivalent melatonin analogue or melatonin receptor agonist to the infant in the breast milk when the infant is suckled sufficient to achieve a chronobiologic effect in the infant. In alternative embodiments, an amount of exogenous melatonin or dose-equivalent melatonin analogue or melatonin receptor agonist sufficient to achieve a chronobiologic effect in the infant is administered directly to the infant, preferably in expressed breast milk including, inter alia, pumped breast milk and banked breast milk, or in formula. The invention also provides methods for achieving an melatonin or dose-equivalent melatonin analogue or melatonin receptor agonist concentration in a pregnant woman's bloodstream that is then conveyed to the fetus through the placental blood circulation.

In a first aspect, the invention provides a method for administering an amount of exogenous melatonin or dose-equivalent melatonin analogue or melatonin receptor agonist to an infant sufficient to achieve a chronobiologic effect, the method comprising the steps of having the infant's mother or wet nurse receive an amount of exogenous melatonin or dose-equivalent melatonin analogue, melatonin precursor or melatonin receptor agonist that produces in the mother's or wet nurse's breast milk a sufficient amount of exogenous melatonin or dose-equivalent melatonin analogue or melatonin receptor agonist to achieve a chronobiologic effect in the infant, wherein the infant is suckled at a time and for a duration effective for the infant to ingest no more than about 1 mg melatonin in the mother's or wet nurse's breast milk at a single feeding, and wherein the infant is suckled at times that are preferably between about 17:00-06:00 hours, more preferably between about 18:00-06:00 hours, and most preferably between about 19:00-06:00. In these embodiments, oral administration of exogenous melatonin or dose-equivalent melatonin analogue, melatonin precursor or melatonin receptor agonist to the mother or wet nurse is preferred. Alternatively, exogenous melatonin or dose-equivalent melatonin analogue, melatonin precursor or melatonin receptor agonist can be administered to a mother or wet nurse using any convenient route or vehicle. Preferably, the amount of exogenous melatonin or dose-equivalent melatonin analogue, melatonin precursor or melatonin receptor agonist administered to the mother or wet nurse is less than about 20 mg, more preferably less than about 10 mg and most preferably less than about 5 mg, provided that the dose produces a concentration of exogenous melatonin or dose-equivalent melatonin analogue or melatonin receptor agonist in the mother's or wet nurse's breast milk sufficient to achieve a chronobiologic effect in the infant, whereby no more than about 1 mg melatonin or dose-equivalent melatonin analogue or melatonin receptor agonist is ingested by the infant in the amount of milk suckled by the infant at a single feeding. In preferred embodiments, the infant falls to sleep within about one hour of suckling said breast milk containing exogenous melatonin or dose-equivalent melatonin analogue or melatonin receptor agonist. Multiple doses can be administered to the infant by suckling, wherein the first dose of exogenous melatonin or dose-equivalent melatonin analogue or melatonin receptor agonist is preferably administered to the infant by suckling at a time that is no more than about 3 hours, more preferably about 2 hours, and most preferably about 0.5-1 hours prior to a time accommodating to the mother's desired bedtime for the infant. A second or any subsequent dose (typically but not necessarily using decreasing amounts) can be taken by suckling the infant up until about 2 hours before the infant's waketime. The infant is preferably less than 1 year old, more preferably less than 6 months old, even more preferably less than 3 months old, and particularly may be a premature infant. A mother or wet nurse will receive exogenous melatonin or dose-equivalent melatonin analogue, melatonin precursor or melatonin receptor agonist at a time wherein the exogenous melatonin or dose-equivalent melatonin analogue or melatonin receptor agonist in her breast milk reaches a concentration sufficient to have a chronobiologic effect on the infant, provided that the infant receives no more than 1 mg exogenous melatonin or dose-equivalent melatonin analogue or melatonin receptor agonist in a single feeding.

In alternative embodiments, an amount of exogenous melatonin or dose-equivalent melatonin analogue or melatonin receptor agonist sufficient to achieve a chronobiologic effect in the infant is administered directly to the infant by adding said exogenous melatonin or dose-equivalent melatonin analogue or melatonin receptor agonist to expressed breast milk including inter alia pumped breast milk and banked breast milk, or to formula. In these embodiments a chronobiologic effect is achieved in the infant by feeding an amount of said breast milk or formula containing exogenous melatonin or dose-equivalent melatonin analogue or melatonin receptor agonist to said infant, wherein the amount of exogenous melatonin or dose-equivalent melatonin analogue or melatonin receptor agonist administered thereby is no more than about 1 mg in a single feeding. Preferably a first dose of exogenous melatonin or dose-equivalent melatonin analogue or melatonin receptor agonist is administered in this aspect of the invention at a time that is no more than about 3 hours, more preferably about 2 hours, and most preferably about 0.5-1 hours prior to a time accommodating to the mother's or primary caregiver's desired bedtime for the infant. In further alternative embodiments about 1 mg of exogenous melatonin or dose-equivalent melatonin analogue or melatonin receptor agonist is administered directly to the infant through other routes, including but not limited to orally/sublingually, rectally and transdermally. In certain aspects exogenous melatonin or dose-equivalent melatonin analogue or melatonin receptor agonist can be administered intravenously, such as in premature infants requiring hospitalization in a neonatal care unit, particularly infants who are treated with phototherapy for hyperbilirubinemia and/or neonatal jaundice, or those treated by exchange transfusion, and any infant having an intravenous line inserted for any medical reason. These aspects of the methods of the invention are particularly advantageous when used with newborn infants and particularly in premature infants (to replicate the chronobiologic effects of melatonin in utero).

In further alternative embodiments, exogenous melatonin or dose-equivalent melatonin analogue or melatonin receptor agonist is administered to a fetus in utero via the pregnant woman. In these embodiments, oral administration to the pregnant woman of exogenous melatonin or dose-equivalent melatonin analogue, melatonin precursor or melatonin receptor agonist is preferred. Alternatively, exogenous melatonin or dose-equivalent melatonin analogue, melatonin precursor or melatonin receptor agonist can be administered to a pregnant woman using any convenient route or vehicle. Regardless of the administration route or vehicle used, administration of not more than about 1 mg exogenous melatonin or dose-equivalent melatonin analogue, melatonin precursor or melatonin receptor agonist to the pregnant woman over a 24-hour period is preferred.

The methods of this invention advantageously improve melatonin or dose-equivalent melatonin analogue, melatonin precursor or melatonin receptor agonist administration to infants over the methods disclosed in the art. Particularly, the methods of this invention permit an infant's circadian rhythms, particularly the infant's wake/sleep rhythm, to be synchronized to accommodate its mother or other caregiver. Moreover, the inventive methods take advantage of the additional salubrious effects of maternal-infant bonding known to occur as a consequence of breast feeding, as well as the well-recognized benefits to the infant of ingesting breast milk. A particular advantage of the methods of the invention is to synchronize an infant's sleep/wake cycle to a desired time that accommodates the mother or primary caregiver, so that a well-rested caregiver will be able to provide better maternal care. Also, intravenous administration is advantageous for hospitalized premature infants, because the levels of exogenous melatonin or dose-equivalent melatonin analogue or melatonin receptor agonist can be precisely adjusted to replicate the in utero environment. A nighttime melatonin signal administered in any of the ways listed above beginning at about 14 hours after the mother's habitual wake-time with a duration of no more than 14 hours mimics the in utero melatonin signal from the pregnant woman to the fetus and may be helpful to all premature infants, particularly those in the hospital and those who are treated with phototherapy. Exogenous melatonin levels or dose-equivalent melatonin analogue or melatonin receptor agonist can be advantageously monitored through an intravenous line as well, although melatonin levels can also be advantageously monitored in saliva and urine samples.

Specific preferred embodiments of the present invention will become evident from the following more detailed description of certain preferred embodiments and the claims.

### DETAILED DESCRIPTION OF THE PREFERRED EMBODIMENTS

The invention provides methods for administering exogenous melatonin or dose-equivalent melatonin analogue or melatonin receptor agonist to a human fetus in utero or to an infant, most preferably a newborn infant, full-term or premature. In certain embodiments for administering exogenous melatonin or dose-equivalent melatonin analogue or melatonin receptor agonist to infants, exogenous melatonin or dose-equivalent melatonin analogue, melatonin precursor or melatonin receptor agonist is contained in breast milk, most preferably breast milk obtained through suckling the infant's mother or wet nurse, wherein the mother or wet nurse is administered exogenous melatonin or dose-equivalent melatonin analogue, melatonin precursor or melatonin receptor agonist prior to suckling the infant. In alternative embodiments, exogenous melatonin or dose-equivalent melatonin analogue or melatonin receptor agonist is administered directly to the infant in a defined dosage, preferably through expressed milk including for example pumped breast milk and banked breast milk, or in formula, to which exogenous melatonin or dose-equivalent melatonin analogue or melatonin receptor agonist is added. Alternatively, the infant can be administered exogenous melatonin or dose-equivalent melatonin analogue or melatonin receptor agonist by routes of administration other than through suckled or expressed breast milk or through formula. In yet further embodiments, melatonin is administered to a fetus in utero by administering melatonin or dose-equivalent melatonin analogue or melatonin receptor agonist to the pregnant woman.

Without wishing to be bound by any hypothesis, and those with skill in the art recognizing how to make and use the claimed invention in the absence of the following considerations, it appears that the primary function of endogenous melatonin production in humans may be to synchronize the late gestational fetus (via the placenta) and to synchronize the sleep/wake cycle of the suckling infant (via breast milk) to the pregnant woman's or mother's circadian cycle, respectively. This is because humans, in contrast to other animals, do not use melatonin to seasonally regulate reproduction, and the light/dark cycle external zeitgeber is in large part sufficient to synchronize the sleep/wake cycle, once the retinohypothalamic tract is functional in the infant after about 3 months of age. Since the capacity to respond to the light/dark cycle does not develop in the infant until about 3 months post-partum, prior to that time this function is provided by maternal melatonin, as long as the ambient light is not overly suppressing the mother's production of melatonin.

The physiological effectiveness of melatonin concentration in breast milk is dependent on an immature liver in the infant that cannot metabolize orally ingested melatonin at adult capacity and efficiency. The liver microsomal enzyme (CPY1A2) that hydroxylates melatonin is only about 1% that of adult activity at birth, about 10% at about age three months and about 50% of adult activity at about age 12 months. Although not yet precisely determined, melatonin bioavailability in the infant is therefore much greater than in adults (where it is about 1-15% of orally-administered melatonin) and begins to decrease at about the same age (about three months) when the infant's endogenous pacemaker synchronizes to the light/dark cycle conveyed via a fully functional suprachiasmatic nucleus and retinohypothalamic tract. At about three months, a functional liver is necessary to metabolize the infant's own considerably high nighttime melatonin levels that begin to appear at this age, so that they do not spill over into the day. However, some infants may still benefit from exogenous melatonin in suckled breast milk until they are about two years old.

In premature infants and in postnatal full-term infants, the liver is even less developed than at 1-3 months of age and thus is much less capable of metabolizing melatonin. It will be understood that their capacity to remove melatonin is limited to urinary excretion and thus is slower and less efficient (and thus melatonin will persist longer in the bloodstream) in these infants. Hence, a lower dose should be administered to these infants, whether through suckling breast milk or through ingestion of formula or expressed milk to which melatonin has been added or from some other direct administration of melatonin to the infant. These considerations do not apply to a fetus in utero, where the pregnant woman's placental circulation both provides and removes melatonin in a dynamic equilibrium depending on her melatonin production and her own liver metabolism of endogenous and exogenous melatonin.

As used herein with regard to the phrase “or dose-equivalent melatonin precursor, melatonin analogue or melatonin receptor agonist,” it will be understood that “dose-equivalent” in intended to mean an amount of a melatonin precursor, melatonin analogue or melatonin receptor agonist that is of a dose level sufficient to provide the same chronobiologic effect as an equivalent dose of melatonin itself.

As used herein, “chronobiologic effect” will be understood to encompass circadian phase-resetting effects or related biological-rhythm effects, on a fetus or infant, particularly a newborn infant and particularly a premature infant. “Chronobiologic effect” will also be understood to encompass melatonin or dose-equivalent melatonin analogue, melatonin precursor or melatonin receptor agonist having a biological night-signaling effect on the fetus or infant, wherein the fetus or infant receives the chemical signal for “night,” regardless of the clock time of day or regardless of light levels the fetus or infant receives from the ambient light/dark cycle.

The present invention contemplates the use of melatonin precursors, melatonin analogues, melatonin agonists and other compounds which mimic melatonin activity, along with or in place of melatonin (N-acetyl-5-hydroxytryptamine) itself. For the purposes of this invention, the term “exogenously administered melatonin” and its variations encompass various formulations of melatonin, melatonin precursors, melatonin receptor agonists (that is, compounds that mimic melatonin's actions) and melatonin analogues. Therefore, whenever reference is made to increasing melatonin levels in plasma or breast milk due to administration of exogenous melatonin, this refers also to increased melatonin levels due to administration of melatonin precursors and also will be understood to encompass increasing equivalent melatonin analogue or melatonin receptor agonist levels due to administration of a melatonin analogue or melatonin receptor agonist, respectively. In these cases, when referring to a melatonin analogue or melatonin receptor agonist, these levels are meant to be bioequivalent to levels of melatonin (that is, to have the same chronobiologic effect as melatonin itself). It will be understood that administration of a melatonin precursor results in increased levels of melatonin itself.

Melatonin can be administered in immediate, sustained or delayed release formulations, or any combination thereof, to achieve a sufficient concentration of melatonin and for a sufficient duration in the mother's bloodstream (for in utero administration) or in suckled milk in the mother's or wet nurse's breast milk in order to achieve a chronobiologic effect in the fetus or suckled infant, whereby a chronobiologic effect in the fetus or infant is achieved. Immediate-release oral and sublingual formulations of melatonin are commercially available, whereas oral delayed release, oral low-dose sustained release and combinations thereof are not commercially available.

Alternate exogenous melatonin formulations, or dose-equivalent formulations of a melatonin analogue or melatonin receptor agonist can also be used to administer exogenous melatonin or dose-equivalent melatonin analogue, melatonin precursor or melatonin receptor agonist to the infant by routes other than suckled or bottled breast milk or formula. In preferred embodiments, exogenous melatonin or dose-equivalent melatonin analogue or melatonin receptor agonist is administered to the infant orally/sublingually, which will be understood to be equivalent modes of administration to an infant. Exogenous melatonin or dose-equivalent melatonin analogue or melatonin receptor agonist can also be administered to an infant in alternative routes including, but not limited to, cremes, salves, lotions, drops, or other topical formulations. Although oral/sublingual administration is preferred, it will be understood that infants are not administered exogenous melatonin or dose-equivalent melatonin analogue or melatonin receptor agonist in age-inappropriate formulations (e.g., capsules).

In preferred embodiments, exogenous melatonin or dose-equivalent melatonin analogue, melatonin precursor or melatonin receptor agonist is administered orally to the mother or wet nurse. Alternate routes include sublingually or in vehicles that are chewed but not swallowed (such as gum or hard candy), by injection, suppository, or transdermally using a patch, lotion, creme, salve or other topical substance or solution. In these embodiments, no more than about 20 mg, more preferably no more than about 10 mg and more preferably no more than about 5 mg exogenous melatonin or dose-equivalent melatonin analogue, melatonin precursor or melatonin receptor agonist is administered to the nursing mother or wet nurse, and no more than about 1 mg in a 24-hour period to a pregnant woman.

Melatonin or dose-equivalent melatonin analogue, melatonin precursor or melatonin receptor agonist is administered at a time chosen to have a chronobiologic effect on an infant's bedtime to accommodate its mother or other caregiver. It will be understood in the art that the terms “an infant's desired bedtime” or “an infant's bedtime to accommodate its mother or other caregiver” indicate the time that the infant's mother, wet nurse or other caregiver desires the infant to be asleep. This may be earlier, later or of longer duration than the mother's sleep cycle, recognizing that the invention provides for infant sleep that can be easily interrupted for purposes of feeding. The infant's desired bedtime may also be during the daytime for a mother who is doing night work, although a consistent day-to-day sleep time for both is preferred. The invention includes the use of melatonin or dose-equivalent melatonin analogue, melatonin precursor or melatonin receptor agonist for treating the mother for a circadian-rhythm sleep or mood disorder, including, but not limited to, pre- or post-partum depression, advanced or delayed sleep phase syndrome and winter depression, while concomitantly using the same doses of exogenous melatonin or dose-equivalent melatonin analogue, melatonin precursor or melatonin receptor agonist taken by the mother as well as additional doses that may be given directly to the infant, to provide a desired effect on the infant's sleep/wake cycle.

The amount of exogenous melatonin or dose-equivalent melatonin analogue or melatonin receptor agonist administered to the infant, most preferably by suckling the infant, or in expressed breast milk including pumped breast milk and banked breast milk, or formula, is sufficient to achieve the desired chronobiologic effect in the infant, most preferably wherein the chronobiologic effect synchronizes the infant's sleep/wake cycle to accommodate the mother or care-giver. As provided in detail herein, the inventive methods provide formula or suckled or expressed breast milk to the infant that comprises exogenous melatonin or dose-equivalent melatonin analogue or melatonin receptor agonist at a concentration wherein the infant ingests no more than about 1 mg exogenous melatonin or dose-equivalent melatonin analogue or melatonin receptor agonist at any single feeding.

In some embodiments, in order to achieve a desired concentration of exogenous melatonin or dose-equivalent melatonin analogue or melatonin receptor agonist in the mother's or wet nurse's suckled breast milk, the mother or wet nurse receives an amount of melatonin or dose-equivalent melatonin analogue, melatonin precursor or melatonin receptor agonist sufficient to achieve a chronobiologic effect on an infant. As provided herein, said sufficient amount is preferably less than about 20 mg, more preferably less than about 10 mg, or even more preferably less than about 5 mg melatonin or dose-equivalent melatonin analogue, melatonin precursor or melatonin receptor agonist, wherein the mother's or wet nurse's breast milk comprises melatonin or dose-equivalent melatonin analogue or melatonin receptor agonist in an amount sufficient to achieve a chronobiologic effect, provided that no more than about 1 mg of exogenous melatonin or dose-equivalent melatonin analogue or melatonin receptor agonist is administered to the infant during suckling at a single feeding. In these embodiments, the mother or wet nurse each day first receives exogenous melatonin or dose-equivalent melatonin analogue, melatonin precursor or melatonin receptor agonist, preferably orally or sublingually, at a time that permits the infant to be suckled no more than about 3 hours, more preferably about 2 hours, and most preferably about 0.5-1 hours prior to a time accommodating to the mother's or primary caregiver's desired bedtime for the infant. As provided herein, an effective way for providing a sufficient concentration of exogenous melatonin or dose-equivalent melatonin analogue or melatonin receptor agonist in breast milk, either for suckling or pumping, is to drain the breast of milk by pumping just prior to the mother or wet nurse receiving exogenous melatonin or dose-equivalent melatonin analogue, melatonin precursor or melatonin receptor agonist; this pumped milk can be saved for later administration to the infant at a time during the day when high levels of melatonin are not desired to be ingested by the infant, whereas any breast milk pumped after the mother or wet nurse has received exogenous melatonin or dose-equivalent melatonin analogue, melatonin precursor or melatonin receptor agonist can be saved for later administration during the night when high levels of melatonin or dose-equivalent melatonin analogue or melatonin receptor agonist are desired to be ingested by the infant. Whether or not the pre-administration pumped breast milk is saved or discarded, draining the breast before administering exogenous melatonin or dose-equivalent melatonin analogue, melatonin precursor or melatonin receptor agonist increases the concentration of exogenous melatonin or dose-equivalent melatonin analogue or melatonin receptor agonist in the breast milk that subsequently accumulates.

Additional doses of immediate-release melatonin or dose-equivalent melatonin analogue, melatonin precursor or melatonin receptor agonist can be taken orally by the mother or wet nurse up to 14 hours after the first dose, provided that the infant ingests no more than 1 mg melatonin or dose-equivalent melatonin analogue or melatonin agonist in a single feeding.

In alternative embodiments, no more than about 1 mg of exogenous melatonin or dose-equivalent melatonin analogue or melatonin receptor agonist is added directly to a defined volume of formula or breast milk, most preferably expressed breast milk including pumped breast milk and banked breast milk, to be ingested by an infant at a single feeding. It will be understood by the skilled worker that breast milk may be ingested by an infant that has been harvested previously (for example, using a breast pump) and maintained, usually by refrigeration or freezing until feeding. In these embodiments, although more than 1 mg of exogenous melatonin or dose-equivalent melatonin analogue or melatonin receptor agonist can be added to said volume of milk, most preferably pumped breast milk, a defined volume can be ingested per feeding by the infant, so that no more than 1 mg of exogenous melatonin or dose-equivalent melatonin analogue or melatonin receptor agonist is ingested at a single feeding. In these embodiments, a first dose of formula or milk containing exogenous melatonin or dose-equivalent melatonin analogue or melatonin receptor agonist, most preferably expressed breast milk including pumped breast milk and banked breast milk, is fed by bottle to the infant no more than about 3 hours, more preferably about 2 hours, and most preferably about 0.5-1 hours prior to a time accommodating to the mother's or primary caregiver's desired bedtime for the infant. In some embodiments, exogenous melatonin or dose-equivalent melatonin analogue or melatonin receptor agonist in amounts greater than 1 mg can be added to a volume of expressed breast milk that is greater than the amount ingested at a single feeding, provided that the resulting expressed milk contains no more than 1 mg melatonin or dose-equivalent melatonin analogue or melatonin receptor agonist in a volume that is ingested by an infant at a single feeding.

Those with skill in the art will understand that it will be advantageous to administer pumped milk to the infant corresponding to the time of pumping (for example, milk pumped during the morning is best given to the infant during the morning, afternoon milk during the afternoon, evening milk during the evening, nighttime milk is administered at night, etc.), and to so identify and use banked breast milk accordingly. In this way the mother's or wet nurse's endogenous melatonin rhythm is communicated to the infant.

Melatonin or dose-equivalent melatonin analogue or melatonin receptor agonist can also be administered to an infant, particularly a newborn infant and particularly a premature infant, by alternative, more “direct” routes and vehicles, including rectal administration by suppository, as well as by topical administration using a patch, lotion, creme, salve, or other topical solution or in liquid form such as drops that are orally/sublingually administered. Premature infants, certainly those in the neonatal intensive care unit, should be administered melatonin or dose-equivalent melatonin analogue or melatonin receptor agonist, including by intravenous administration, beginning at 14 hours after the mother's habitual wake-time with a duration of no more than 14 hours, so as to mimic in utero conditions. Advantageous combinations of said administration regimes can also be employed; for example, melatonin or dose-equivalent melatonin analogue or melatonin receptor agonist in a form applied to the mother's or wet nurse's nipple, immediately before suckling the infant, can be used to achieve oral/sublingual administration directly to an infant. Similarly, artificial suckling means such as a pacifier can be coated, embedded or otherwise supplied with melatonin or dose-equivalent melatonin analogue or melatonin receptor agonist for direct oral/sublingual administration, provided these embodiments are configured to deliver no more than about 1 mg exogenous melatonin or dose-equivalent melatonin analogue or melatonin receptor agonist to the infant in a defined amount of time (i.e., equivalent to a feeding) per single usage.

Used as disclosed herein, the methods of this invention result in an infant falling to sleep within about 1 hour after ingesting exogenous melatonin or dose-equivalent melatonin analogue, melatonin precursor or melatonin receptor agonist. In additional embodiments, an infant who awakens after initially falling asleep is further suckled by the mother or wet nurse, wherein the concentration of exogenous melatonin or dose-equivalent melatonin analogue or melatonin receptor agonist in the mother's or wet nurse's breast milk remains at a concentration sufficient to have a chronobiologic effect provided that no more than about 1 mg exogenous melatonin or dose-equivalent melatonin analogue or melatonin receptor agonist is delivered to the suckled infant at each feeding.

For administration of exogenous melatonin or dose-equivalent melatonin analogue or melatonin receptor agonist to a fetus in utero via the mother, oral or sublingual administration of exogenous melatonin or dose-equivalent melatonin analogue, melatonin precursor or melatonin receptor agonist to its pregnant mother is preferred. Alternatively, exogenous melatonin or dose-equivalent melatonin analogue, melatonin precursor or melatonin receptor agonist can be administered to the pregnant woman using any convenient route or vehicle. Regardless of the administration route or vehicle used, no more than physiological amounts of exogenous melatonin or dose-equivalent melatonin analogue or melatonin receptor agonist are required, in view of the placental connection of the fetus to the maternal bloodstream. Thus, it is expected that the blood concentration of melatonin in the fetus with be equivalent to the melatonin concentration in the mother. Preferably, no more than about 1 mg exogenous melatonin or dose-equivalent melatonin analogue, melatonin precursor or melatonin receptor agonist is administered to the mother in a 24-hour period to achieve a chronobiologic effect in the fetus, although the 1 mg can be delivered in more than one divided dose. In this embodiment, exogenous melatonin or dose-equivalent melatonin analogue, melatonin precursor or melatonin receptor agonist is first administered to the pregnant woman about 14 hours after her wake-up time.

It will be understood by those with skill in the art that, as a consequence of the existence of the melatonin phase response curve (PRC; first disclosed in U.S. Pat. No. 5,242,941, issued Sep. 7, 1993 and incorporated herein by reference), any administration of exogenous melatonin or dose-equivalent melatonin analogue, melatonin precursor or melatonin receptor agonist will potentially cause a phase shift (unless said administration time falls within one of melatonin PRC's “dead zones,” that is, zones of relative insensitivity to the effects of exogenous melatonin, or an additional dose is taken at a time appropriate to counter-balance the effects of melatonin administration), or at a sufficiently-high dose (about 15-20 mg) that stimulates phase-advance and phase-delay zones equally. Melatonin or dose-equivalent melatonin analogue, melatonin precursor or melatonin receptor agonist administration to an infant via the mother or wet nurse performed in ignorance of such effects on the melatonin PRC runs the risk of causing inappropriate phase shifts especially in the mother or wet nurse. Thus, it is evident from the present disclosure and the teachings of U.S. Pat. No. 5,242,941 that an individual's melatonin PRC must be understood and taken into account whenever exogenous melatonin or dose-equivalent melatonin analogue, melatonin precursor or melatonin receptor agonist is administered to a human.

Thus, exogenous melatonin or dose-equivalent melatonin precursor, precursor, melatonin analogue or melatonin receptor agonist administration to a pregnant or suckling mother is advantageously timed to coincide with a “dead zone” in the mother's melatonin PRC, e.g. between about CT 14-16, unless a phase shift is actually desired. It will be understood that administration of sufficiently high doses of melatonin or dose-equivalent melatonin precursor, melatonin analogue or melatonin receptor agonist to the mother or wet nurse at times other than times in the dead zone will avoid a chronobiologic effect because that amount (about 15-20 mg) will persist in the mother's or wet nurse's bloodstream for a time that affects both the phase-advance as well as the phase-delay portions of the phase-response curve, most preferably to an equivalent degree to effect no phase shifting. Alternatively, exogenous melatonin or dose-equivalent melatonin precursor, melatonin analogue or melatonin receptor agonist can be administered to the mother or wet nurse at time to cause a phase delay that balances out any phase advance due to prior ingestion of melatonin or dose-equivalent melatonin precursor, melatonin analogue or melatonin agonist administered on the phase-advance zone of the melatonin PRC. Melatonin, melatonin analogue, melatonin precursor or melatonin receptor agonist administered at other times sufficient to cause an equal phase delay by repeated dosing includes administration in the middle of the night and/or early morning. Alternatively, a phase shift can be avoided by administering an appropriate sustained-release formulation, or delayed-release or delayed release/sustained release formulation, or combination thereof, taken in the evening that sufficiently elevates levels throughout the night.

The following examples describe certain specific embodiments of the invention. However, many additional embodiments not described herein nevertheless fail within the spirit and scope of the present invention and claims.

### EXAMPLE 1

### Detection of Melatonin Levels in Human Blood Plasma and Milk

The amount of melatonin in human blood (from an infant or mother/wet nurse) can be estimated from the amount in saliva, which is much easier to collect from an infant than venous blood. It will be understood that saliva is most advantageously colleted after wiping the infant’ mouth to dryness, to ensure that the accuracy of the analysis is not compromised by melatonin from mother's milk. Saliva samples (0.1-0.4 mL) were collected in absorbent material and then centrifuged and frozen for later analysis by radioimmunoassay using a commercially-available kit according to the manufacturer's instructions (Melatonin (Direct Saliva) RIA kit, ALPCO, Inc., Salem, N.H.).

### EXAMPLE 2

The sleep/wake circadian rhythm of a newborn infant was manipulated using administration of exogenous melatonin to a mother who fed the infant only by suckling or by her pumped breast milk according to the instant invention. The following log includes nighttime sleep data of the infant taking nothing else by mouth except thrush medication during about weeks 2-4. A dose to the mother of 1.8-12 mg (usually 3 mg) in the evening, sometimes followed by 1-2 mg in the middle of the night or even 0.5 mg towards the end of the night resulted in better sleep for both mother and newborn with no reduction of actual breast-feeding time during the night and steady weight gain in the baby (quick return to near pre-pregnancy weight in the mother, probably due to good sleep in the mother). Furthermore, there appeared to be less evening/nighttime colic in the infant. Moreover, the invention appeared to accelerate development of the infant's independent ability to synchronize to the ambient light/dark cycle. All melatonin used in this Example was an immediate-release formulation taken by the mother orally.

Week 1

Sunday (10 days post-partum, first day of exogenous melatonin administration in breast milk):
At 20:50 mother took 3 mg melatonin orally. At 21:20 breast-fed baby. After feeding, for the first time in the 10 days since birth, baby slept between 22:00 and 04:00, at which time he was easily awakened for his next feeding (mother slept well for the first time and awoke due to breast discomfort due to too much milk). Mother and baby slept for a few more hours.
   Monday:
At 19:45, mother took 1.05 mg. Baby fed at 20:05 and fell asleep. He awoke at 21:30 and fed again. Both slept from 22:15 until 02:00, and then slept or were eating every 90 minutes until morning.
   Tuesday:
At 20:30, mother took 0.9 mg and fed at 9. Baby fell asleep at 10:30 and awoke at 02:00.
   Wednesday:
At 20:45, mother took 3 mg. Baby fed only one breast after starting feeding at 21:00 and both fell asleep at 21:30. Baby fed at 01:30 and 02:00 (second breast). Both slept until 05:30, fed, and then slept until 07:15.
   Thursday.
Mother took 3 mg at 20:45. Fell asleep 22:30-02:30. Then slept until 05:30.
   Friday and Saturday:
Mother took 3 mg at 20:30 and they fell asleep by 22:15, awakened around 02:00, and then 05:00 for feedings, sleeping until 08:00.
   Week 2
   Sunday.
Mother took 3 mg at 20:30 but ate immediately after. Fell asleep at 20:00 but awoke at 23:00, 01:30 and 04:30, and both fell back to sleep but not as quick and mother was awake 04:30-06:30
   Monday
Mother took 3 mg at 20:30. Asleep at about 21:30. Nursed at 01:30, 04:30, 06:20 and 07:30.
   Tuesday:
Mother took 3 mg at 20:30. Baby fell asleep at 21:15 until 01:00. Fed and went back to sleep at 04:30 and 06:30 and 07:15.
   Wednesday
Mother took 3 mg at 20:30. Fell asleep at 21:30 and slept until 01:00, 03:30 and 05:30, feeding and going back to sleep well.
   Thursday:
Mother took 3 mg at 20:30, baby fell asleep 21:45. Baby woke and fed at 01:00, 04:00 but was fussy with straining to make a bowel movement, slept until 06:15.
   Friday
Mother took 3 mg at 20:30. Baby fell asleep at 22:00. Baby awake at 01:30 and 02:30 and 03:30 and 05:30 and 06:00 and 07:30 (but mother did not have her water container and was dehydrated). Baby asleep (mostly) between 07:00-11:00.
   Saturday:
Mother took 3 mg at 20:30. Baby awoke at 21:45, 01:00, 03:00, 05:00 and 06:30.
   Week 3
   Sunday:
Mother took 3 mg at 15:00. Not sure if baby was more sleepy but it did seem that we were keeping him up until he fell asleep very quickly 21:30. Slept until 23:45, 01:00, 03:00, 05:00 and 06:00. Fussy after this but slept from 07:30-09:00.
   Monday
Mother took 3 mg at 17:20. Neither baby nor mother appeared to be sleepy for the few hours after the melatonin dose. Mother felt like falling asleep about 21:00. Both baby and mother fell asleep at 21:45. Baby slept until 01:30 and then 05:30 and nursed on both sides which was unusual. Baby awoke at 07:00.
   Tuesday:
Mother took 3 mg at 17:00. Neither baby nor mother appeared to be sleepy for the few hours after the melatonin dose. Baby fell asleep at 21:15 and up at 01:00, 04:00, 05:00 and 06:30. However, mother was up from 01:30-03:30, but felt good in the morning and felt particularly good in the afternoon. Baby's weight gain is excellent on melatonin, better than before melatonin. Baby sleeping well between feedings.
   Wednesday:
Mother took 1.8 mg at 17:45. Baby fell asleep at 21:20 and mother at 21:40. Baby awoke at 01:00, 04:00, 05:00, 06:00 and 07:30. Mother up between 01:00 and 03:30.
   Thursday:
Mother took 3 mg at 17:00. Baby fell asleep at 21:00 until 24:00, and then until 03:00, 05:30 and 07:30. Mother tired in the afternoon and awake from 03:00-04:30 but energetic in the morning.
   Friday:
Mother took 3 mg at 20:30. Baby fell asleep at 22:00. Baby awake at 01:00, 03:30, 06:00 and 07:30. Mother was not awake in middle of the night, except for about 15 minutes.
   Saturday:
Mother took 2 mg at 19:15. Mother and baby fell asleep at 21:30, fed at 01:10, 03:00, 04:00, 05:00 and 06:00 and then he slept all morning.
   Week 4
   Sunday:
Mother took 2 mg at 19:30. Baby fell asleep at 21:30, 01:30, 04:00, and 05:45. Baby slept until 08:00.
   Monday:
Mother took 2 mg at 19:30. Baby fell asleep at 22:15, awoke at 01:10, 03:00 and 04:15, but fussy until 05:00 and slept until 06:30 and 08:30.
   Tuesday
Mother took 3 mg at 17:50. Baby fell asleep at 21:40 (mother at 22:00), fed and went right back to sleep at midnight, 01:30, 03:00, 05:40 and 08:10.
   Wednesday:
Mother took 0.6 mg at 14:50 and 3 mg 19:30. Baby fell asleep at 21:45 and 22:00, although mother could have gone to sleep an hour earlier. Awoke at 00:30, 03:00, 04:00 and fussy until 06:30, then slept until awakened at 08:00.
   Thursday:
Mother took 3 mg at 17:50. Baby went to sleep at 21:40 and mother could have gone to sleep earlier. Woke at 23:45, 00:50, 03:00, 03:30, 04:00, 05:30 and 06:30, because of fussiness between 03:00-04:00 and mother awake from 03:00-04:30.
   Friday:
Mother took 3 mg at 20:40. Baby asleep at 22:10. Baby awoke at 01:10, 03:07, 05:50 and 06:30. However, was colicky during the evening.
   Saturday:
Mother took 3 mg at 17:50. Slept at 21:50. Baby awoke at 02:15, 04:30, 05:42 and 07:30. Mother was awake at 04:00-05:00 and took 2 mg at 05:00 and was sleepy in the morning.
   Week 5
   Sunday:

Mother took 3 mg at 17:30. Less colic. Baby went to sleep at 21:45. Baby awoke at 01:20, 03:00, 04:30, 05:40 and 07:15. Mother slept all night and woke at 05:30.

Monday:

Mother took 3 mg at 17:00. In bed at 21:45. Baby awoke at 00:45, 02:30, 05:00 and 07:15. Colic was about 40% better than its worst.
   Tuesday:
Mother took 3 mg at 17:00. Baby asleep at 21:30, 01:10, 03:20, 05:18 and 08:19. Much better night but fussy in the evening.
   Wednesday:
Mother took 3 mg at 21:45. Asleep at 22:40, baby awoke at 01:41, 03:02, 04:43, 05:15, 06:30 and 08:00.
   Thursday:
Mother took 3 mg at 17:30. Baby asleep at 21:30, baby awoke at 01:00, 03:35, 05:00, 06:20 and 07:30.
   Friday:
Mother took 3 mg at 18:25. Baby asleep at 09:30, 00:30, 03:10, 05:40 and 08:00. Mother falling asleep at 09:00, up between 00:30 and 03:30.
   Saturday:
Mother took 3 mg at 21:30. Baby fell asleep at 22:00. Baby awoke at 00:40, 03:07, 05:20 and 07:30. Mother slept the whole night.
   Week 6
   Sunday:
Mother took 3 mg at 17:30. Baby asleep at 21:45. Baby awoke at 23:40, 03:21, 05:06, 06:20 and 07:30. Best night ever, except that mother was up from 05:06.
   Monday:
Mother took 2 mg 17:30. Baby asleep by 21:45. Awoke at 23:50, 03:08 and mother took 1 mg (mother awake for 30 minutes), 04:40, 06:37 and 09:00.
   Tuesday:
Mother took 2 mg at 19:30. Baby napped from 20:30-21:00 and then fell asleep at 21:30. Baby awoke at 00:45, 01:42, 03:04 (at which time mother took 1 mg), 05:48 and 07:15. Baby awake at 03:00 for a half-hour, otherwise mother slept fine but got up at 06:00.
   Wednesday:
Mother took 3 mg at 19:10. Baby asleep at 21:30. Baby awake at 02:00, 04:45 and 07:54. Mother took 1 mg at 02:00.
   Thursday:
Mother took 3 mg at 19:30. Baby asleep 21:45, 00:30, 06;11 and 08:00. Mother did not take any melatonin during the night and was up from 04:00-05:00.
   Friday:
Mother took 3 mg at 07:15. Baby asleep at 21:40. Baby awoke at 00:18, 04:00 (a little fussy), 06:15 and 08:45. Mother awake from 04:00-05:00.
   Saturday
Mother took 3 mg at 21:30. Baby asleep at 22:10. Baby awoke at 00:06, 01:10, 03:50, 04:54 and 06:30 and then at 08:00. Mother took 0.6 mg at 03:50 and was up until next feeding at 04:40.
   Week 7
   Sunday:
Baby asleep at 21:50. Baby awoke at 23:30, 02:30 (mother took 1 mg and up for an hour), 04:36, 05:49 and 06:55.
   Monday:
Mother took 3 mg at 19:00. Baby awoke at 19:15, 21:00, 21:15, 23:20, 01:00 (mother took 2 mg at 01:00 and up from 04:00-05:15), 02:20, 04:17, 06:12 and 06:38.
   Tuesday:
Mother took 3 mg at 19:00. Baby asleep at 21:15. Baby awoke at 00:10, 02:17, 04:14, 06:06 and 07:15. Mother took 2 mg at 02:17, awake for an hour and pumped milk.
   Wednesday:
Mother took 3 mg at 21:30. Baby asleep at 21:20. Baby awake at 23:44, 02:15, 04:20, 06:00 and 06:40. Mother pumped breast milk at midnight and was up for about 45 minutes. Mother took no additional melatonin.
   Thursday:
Mother took 3 mg at 18:40. Baby asleep at 21:50. Baby awoke at 02:00, 05:42 and 06:30. Mother was awake from 02:00-04:30 and pumped beast milk and took 0.6 mg at 04:00. No baby bowel movements at night for past week, suggesting that the melatonin treatment caused early synchronization even if melatonin is stopped at this phase of his development
   Friday:
Mother took 3 mg at 19:30. Baby asleep at 22:00. Baby awoke at 01:37, 04:51 and 06:00.
   Saturday:
Mother took 3 mg at 19:30. Baby asleep at 09:30. Baby awoke at 00:40, 03:50, 04:22, 05:37 and 07:00. Mother took 0.4 mg at 04:22 and up for 20-30 minutes.
   Week 8
   Sunday:
Mother took 3 mg at 20:30. Baby asleep at 21:45. Baby awoke at 01:12, 05:04 and 08:00. Mother up from 05:00-06:15 (and pumped breast milk).
   Monday:
Baby asleep at 21:00. Mother asleep at 21:30. Baby awoke at 00:00, 02:15, 04:45 and 06:00. Mother slept well and did not pump breast milk.
   Tuesday:
Mother took no melatonin. Baby asleep at 22:00. Baby awoke at 02:09, 04:40, 06:00 and 08:40. Mother awake from 04:40 to after 06:00 and pumped breast milk.
   Wednesday:
Baby asleep at 22:00 and mother asleep at 22:30. Baby awoke at 00:15, 02:07, 04:15, 04:41 (fussy) and then 07:45. Seemed that the longest sleep shifted to a slightly later time.
   Thursday:
Baby asleep 21:45. Baby awoke at 00:06, 02:30, 03:00 (fussy), 05:30 and 07:30.
   Friday:
Baby asleep at 22:10. Baby awoke at 01:30, 04:30, 07:30 and another nap later in the morning. Mother slept from the same time and well.
   Saturday:
Baby asleep at 22:00. Baby awoke at 01:52, 04;14, 05:20, 06:42 and 09:15. Mother slept well from 22:15.
   Week 9
   Sunday:
Baby asleep at 09:40. Baby and mother awoke at 01:12 (mother pumped breast milk) 03:41, 05:26 and 06:37.
   Monday:
Baby asleep at 22:00. Baby awoke at 12:50, 03:00, 04:14, 05:38 and 06:48 and slept until 8:15.
   Tuesday:
Received daytime pumped milk in morning and was alert afterwards. Baby asleep at 22:00. Baby awoke at 01:33, 04:05, 06:24 and 08:15.
   Wednesday:
Baby asleep at 21:40. Baby awoke at 01:07, 05:38, 06:27 and was awakened at 08:10.
   Thursday:
Fed at 16:00 and then for a couple of minutes before 17:40. Received 45 mL of nighttime pumped breast milk and slept well all night.
   Friday:
Asleep at 21:45. Awoke at 03:00, 03:55, 05:48 and 08:15. Fed at 06:00. Mother slept well.
   Saturday:
Asleep at 09:30. Awoke at 03:15, 06:11 and 08:15. Mother slept well.
   Week 10
   Sunday:
Asleep at 22:20, but because mother got into bed late. Awoke at 02:00, 04:38, 05:48 and 08:15.
   Monday:
Asleep at 21:35. Awoke at 01:37, 03:28 and 06:50.
   Tuesday:
Asleep at 21:15. Awoke at 00:00, 03:25, 06:00 and 07:30. Been napping until 02:30 and not eating much during this time.
   Wednesday:
Asleep at 21:35. Awoke at 03:15, 06:44 and 08:10.
   Thursday:
Asleep at 09:00. Awoke at 00:35, 03:09, 05:27 and 07:30. Took 3 mg at 00:19.
   Friday:
Asleep at 09:30. Awoke at 00:00, 02:52, 05:14, 06:50 and 08:35.
   Saturday:
Asleep at 21:15. Awoke at 01:33, 04:50, 05:25, 06:47 and 09:00.
   Week 11
   Sunday:
Asleep at 21:25. Awoke at 01:38, 03:20, 06:33 and 07:30.
   Monday:
Asleep at 21:30. Awoke at 23:06, 02:34, 05:06, 06:30 and 08:00.
   Tuesday:
Asleep at 20:45. Awoke at 21:25, 01:46, 04:40, 06:15 and 07:24.
   Wednesday:
Asleep at 21:22, 22:00, 23:39, 04:14, 05:36 and 08:10.
   Thursday:
Asleep at 21:15. Awoke at 21:45, 02:53, 04:45, 06:53 and 07:30.
   Friday:
Asleep at 21:35. Awoke at 03:22, 06:25 and 08:00.
   Saturday:
Mother took 12 mg melatonin at 19:00. Asleep at 22:00. Awoke at 00:44, 04:30 and 06:50.
   Week 12
   Sunday:
Asleep at 22:15. Awoke at 00:35, 03:13, 04:00, 06:07 and 08:17.
   Monday:
Asleep at 21:27. Awoke at 01:37, 04:0, 05:44 and 08:10.
   Tuesday:
Asleep at 20:30. Awake at: 11:20, 03:20, 05:55, 06:20 and 08:30.
   Wednesday:
Asleep at 21:30. Awake at 23:36, 02:57, 03:50, 05:11, 07:00 and 07:45. Mother awake from 02:47-04:30.
   Thursday:
Mother took 3 mg melatonin at 19:24. Asleep at 21:35. Awake at 01:49, 03:35, 06:00 and 07:45.
   Friday:
Asleep at 22:30. Awake at 02:33, 03:41, 06:00, 06:25 and 07:08.
   Saturday:
Asleep at 9:10. Awake at: 9:10, 9:50, 12:30, 3:00, 5:32, 7:00, 8:50
   Week 13
   Sunday:
Mother took 3 mg melatonin at 19:30.
Asleep at 21:30. Awake at 03:30, 05:45 and 07:20.
   Monday:
Asleep at 21:45. Awake at 00:49, 04:02, 05:51 and 08:14.
   Tuesday:
Asleep at 21:15. Awake at 22:32, 00:28, 01:18, 02:41,0 4:09, 06:15 and 07:45. Very fussy in general during the night probably due to immunizations.
   Wednesday:
Mother took 3 mg melatonin at 19:30. Asleep at 21:05, awake at 21:45, 00:26, 03:55, 06:41 and 08:11.
   Thursday:
Asleep at 21:44. Awake at 01:27, 04:35, 05:08 and 07:15.
   Friday:
Mother took 3 mg melatonin at 19:30. Asleep at 21:00. Awake at 23:45, 00:30, 03:45 and 05:56.
   Saturday:
Took 3 mg at 19:40. Asleep at 20:45. Awake at 21:30, 00:09, 01:26, 04:45, 06:50 and 08:03.
   Week 14
   Sunday:
Asleep at 20:45. Awake at 09:30, 02:42, 05:24, 06:41 and 08:00.
   Monday:
Asleep at 20:10. Awake at 20:45, 00:05, 03:26, 05:56 and 07:18.
   Tuesday:
Asleep at 21:38. Awake at 00:04, 04:16, 05:28 and 07:46.
   Wednesday:
Asleep at 20:45. Awake at 21:00, 01:23, 03:26, 05:51, 07:14 and 08:10 (lots of interruptions).
   Thursday:
Asleep at 19:15 until 19:45. Asleep again at 21:45. Awoke at 00:12, 01:57, 03:17, 04:36 and 06:57.
   Friday:
Asleep at 21:15. Awoke at 00:41, 04:21, 05:30 and 07:20.
   Saturday:
Asleep at 20:45 until 21:11. Awoke at 21:45, 00:12, 01:50, 04:21, 05:27, 06:18 and 07:45.
   Week 15
   Sunday:
Asleep at 20:15. Awake at 20:45 until 21:15. Awoke at 21:45, 22:15, 01:07, 04:21, 05:56 and 08:45.
   Monday:
Asleep at 21:36. Awake at 01:59, 05:18, 06:03 and 07:56.
   Tuesday:
Asleep at 21:30. Awoke at 10:15, 01:58, 03:22, 06:30 and 08:52.
   Wednesday:
Asleep at 21:40. Awoke at 23:59, 02:45, 04:29, 06:22 and 08:12.
   Thursday:
Asleep at 21:40. Awoke at 02:30, 04:01, 04:59, 06:55 and 07:45.
   Friday:
Mother took 3 mg melatonin at 19:30. Asleep at 21:35. Awake at 00:30, 04:25, 06:51, 0:730, and 08:31. Mother awake 04:25-06:00.
   Saturday:
Mother took 3 mg melatonin at 19:30. Asleep at 21:50. Awake at 00:55, 03:33, 04:41, 06:43 and 08:17. Mother awake 04:41-05:40.
   Week 16
   Sunday:
Mother took 3 mg melatonin at 19:430. Asleep at 20:56. Awake at 23:12, 01:42, 03:03, 05:22, 06:03,0 07:22 and 08:50. Mother awake 05:22-06:22.
   Monday:
Mother took 3 mg melatonin at 19:30. Asleep at 21:00. Awake at 23:05, 00:20, 04:04 (bowel movement and fussy for an hour), 05:44 and 08:11.
   Tuesday:
Took 3 mg at 19:30. Asleep at 21:25. Awake at 00:07, 02:15, 04:11, 05:55 and 08:30.
   Wednesday:
Mother took 3 mg melatonin at 19:30. Awoke at 21:15, 21:30, 01:23, 05:04 and 07:30 (awakened by mother).
   Thursday:
Mother took 3 mg melatonin at 19:30. Asleep at 20:45-21:15 (awakened by mother). Awoke at 00:47, 04:27, 05:50 and 07:11.
   Friday:
Mother took 3 mg melatonin at 21:00. Asleep at 21:20. Awake at 01:27, 04:11, 05:44, 06:18, 07:30 and 07:50.
   Saturday:
Mother took 3 mg melatonin at 19:30. Asleep at 21:30. Awake at 01:15, 01:25, 03:51, 06:00 and 07:30.
   Week 17
   Sunday:
Took at 3 mg at 19:30. Asleep at 21:00. Awake at 00:00, 01:48, 02:30 (took 0.4 mg), 05:50 and 07:00.
   Monday:
Asleep at 20:50. Awake at 21:05, 23:27, 02:05, 05:45, 06:50 and 08:15.
   Tuesday:
Asleep at 21:00. Awake at 21:30, 00:13, 02:03, 04:14, 06:07 nursed on and off until 07:00 and then slept until 07:45.
   Wednesday:
Asleep at 21:18. Awake at 23:17, 01:22, 03:20, 04:22, 06:11 and 07:45.
   Thursday:
Asleep at 21:20. Awake at 0!:30, 04:06, 07:10 and 08:00.
   Friday:
Asleep at 20:50. Woke him up at 21:15. He then awoke at 00:18, 01:22, 04:15 and 07:14.
   Saturday:
Asleep at 21:15. Woke him up at 22:00. He stayed awake until 22:30 and awoke at 03:52, 05:53, 07:21 and 08:30.
   Week 18
   Sunday:
Asleep at 21:00. Awoke at 21:35, 00:07, fussy until next feeding at 00:44. Awoke at 03:04, 05:05, 06:20, 07:20 and 09:40.
   Monday:
Asleep at 21:45. Awoke at 21:45, 00:45, 04:37 and 06:17.
   Tuesday:
Asleep at 21:20. Awake at 23:42, 02:30, 03:52 and 06:26 (Mother awake from 02:30-04:30).
   Wednesday:
Asleep at 20:15. Awoke at 20:55 and fell asleep at 21:30. Awoke at 00:34, 02:20, 05:16 and 06:41.
   Thursday:
Asleep at 32:32. Awoke at 23:55, 02:48, 03:45 (Mother moved infant), and 06:02.
   Friday:
Asleep at 20:50. Mother moved and infant awakened at 21:15. Fell back asleep at 22:14. Awake at 00:35, 01:47, 03:04, 05:06, 06:46 and 07:32.
   Saturday:
Asleep at 22:15. Awoke at 22:48, 01:26, 03:17, 05:41 and 07:36.
   Week 19
   Sunday:
Asleep at 21:58. Awoke at 22:28, 01:41, 04:09, 04:50 (Mother moved), 06:20, 06:42 and 09:00.
   Monday:
Asleep 20:45-21:15 and again at 22:45. Awoke at 00:52, 02:21, 03:05, 05:08 and 08:42.
   Tuesday:
Asleep at 21:30. Awoke 01:00 03:19 (stayed awake until 04:23), 06:04, 07:20 and 08:50.
   Wednesday:
Asleep from 20:45-21:20. Awoke at 21:20, 02:24, 04:11 (until 04:40) and 07:00.
   Thursday:
Asleep at 21:40. Awoke at 02:12, 00:56, 05:10 and 07:30.
   Friday:
Asleep at 21:49. Awoke at 00:47, 03:00, 06:00 and 07:00.
   Saturday:
Asleep at 21:18. Awoke at 22:47, 01:11, 07:48 and 08:50.
   Week 20
   Sunday:
Asleep in crib from 21:35-23:00. Awoke at 23:00, 02:06, 06:00 and 08:41.

All references cited herein are incorporated by reference. In addition, the invention is not intended to be limited to the disclosed embodiments of the invention. It should be understood that the foregoing disclosure emphasizes certain specific embodiments of the invention and that all modifications or alternatives equivalent thereto are within the spirit and scope of the invention as set forth in the appended claims.

We claim:

1. A method for achieving a chronobiologic effect on a circadian rhythm in an infant to accommodate its mother or other caregiver, the method comprising the steps of:
a) having the infant's mother or wet nurse receive an amount of melatonin or dose-equivalent melatonin precursor, melatonin analogue or melatonin receptor agonist to produce a concentration of melatonin or dose-equivalent melatonin analogue or melatonin receptor agonist in the mother's or wet nurse's breast milk sufficient to have a chronobiologic effect in the infant provided that no more than about 1 mg melatonin or dose-equivalent melatonin analogue or melatonin receptor agonist is administered to the infant from suckling at a single feeding; and
b) suckling the infant at a time wherein the mother's or wet nurse's breast milk contains an amount of melatonin or dose-equivalent melatonin analogue or melatonin receptor agonist greater than the mother's or wet nurse's endogenous melatonin produced physiologically in her breast milk and at a time and for a duration sufficient to administer no more than about 1 mg melatonin or dose-equivalent melatonin analogue or melatonin receptor agonist to the infant from suckling at a single feeding, wherein a chronobiologic effect in the infant is achieved.

2. The method of claim 1 wherein the circadian rhythm is the sleep/wake cycle circadian rhythm.

3. The method of claim 1 wherein the infant is suckled at a time that is from about 1 hour to about 3 hours prior to a sleep onset time for the infant that accommodates its caregivers.

4. The method of claim 1 wherein the mother or wet nurse receives less than about 20 mg exogenous melatonin or dose-equivalent melatonin precursor, melatonin analogue or melatonin receptor agonist, provided that the mother's or wet nurse's breast milk contains thereafter a concentration of melatonin or dose-equivalent melatonin analogue or melatonin receptor agonist sufficient to have a chronobiologic effect in the infant and provided that no more than about 1 mg melatonin or dose-equivalent melatonin analogue or melatonin receptor agonist is administered to a suckled infant in a single feeding.

5. The method of claim 1 wherein the mother or wet nurse receives less than about 10 mg exogenous melatonin or dose-equivalent melatonin precursor, melatonin analogue or melatonin receptor agonist, provided that the mother's or wet nurse's breast milk contains thereafter a concentration of melatonin or dose-equivalent melatonin analogue or melatonin receptor agonist sufficient to have a chronobiologic effect in the infant and provided that no more than about 1 mg melatonin or dose-equivalent melatonin analogue or melatonin receptor agonist is administered to a suckled infant in a single feeding.

6. The method of claim 1 wherein the mother or wet nurse receives less than about 5 mg exogenous melatonin or dose-equivalent melatonin precursor, melatonin analogue or melatonin receptor agonist, provided that the mother's or wet nurse's breast milk contains thereafter a concentration of melatonin or dose-equivalent melatonin analogue or melatonin receptor agonist sufficient to have a chronobiologic effect in the infant provided that no more than about 1 mg melatonin or dose-equivalent melatonin analogue or melatonin receptor agonist is administered to a suckled infant in a single feeding.

7. The method of claim 1, wherein the mother or wet nurse receives melatonin or dose-equivalent melatonin precursor, melatonin analogue or melatonin receptor agonist orally, sublingually, rectally or transdermally.

8. The method of claim 1, wherein the infant falls asleep after ingesting melatonin or dose-equivalent melatonin analogue or melatonin receptor agonist from being suckled by the mother or wet nurse.

9. The method of claim 1, further comprising the step of further suckling the infant upon awakening after initially falling asleep, wherein the mother's or wet nurse's breast milk contains a concentration of melatonin or dose-equivalent melatonin analogue or melatonin receptor agonist sufficient to have a chronobiologic effect in the infant provided that no more than about 1 mg melatonin or dose-equivalent melatonin analogue or melatonin receptor agonist is administered to the suckled infant at each feeding.

10. The method of claim 1 wherein the mother or wet nurse receives melatonin or dose-equivalent melatonin precursor, melatonin analogue or melatonin receptor agonist at a time that is within one of the dead zones of the mother's or wet nurse's melatonin phase response curve.

11. The method of claim 1 wherein the mother or wet nurse has a first administration of melatonin or dose-equivalent melatonin precursor, melatonin analogue or melatonin receptor agonist on the phase advance zone of her phase response curve, wherein the same mother or wet nurse thereafter has a second or subsequent administration of melatonin or dose-equivalent melatonin precursor, melatonin analogue or melatonin receptor agonist, on the phase-delay zone of her melatonin phase response curve that has the opposite effect on her circadian rhythms as the first administration so as to balance the circadian rhythm phase-advancing effect from her first dose of melatonin or dose-equivalent melatonin precursor, melatonin analogue or melatonin receptor agonist, wherein the phase of her circadian rhythms is unaffected by her receiving melatonin or dose-equivalent melatonin precursor, melatonin analogue or melatonin receptor agonist.

12. The method of claim 1 wherein the mother or wet nurse receives less than 20 mg melatonin or dose-equivalent melatonin precursor, melatonin analogue or melatonin receptor agonist so that the phase-advancing and phase-delaying effects on the mother's or wet nurse's circadian rhythms are balanced and the mother or wet nurse experiences no net circadian rhythm phase-shifting effects.

13. The method of claim 12 wherein the melatonin or dose-equivalent melatonin precursor, melatonin analogue or melatonin receptor agonist is administered in a sustained-release formulation.

14. The method of claim 1 wherein the melatonin or dose-equivalent melatonin precursor, melatonin analogue or melatonin receptor agonist is administered to the mother or wet nurse in an immediate, sustained, or delayed release formulation, or effective combinations thereof.

15. The method of claim 1 wherein the infant is a premature infant.

16. The method of claim 1, wherein the mother or wet nurse receives melatonin or dose-equivalent melatonin precursor, melatonin analogue or melatonin receptor agonist in two or more doses at different times, wherein the doses are administered within a time interval of less than about 14 hours.

17. The method of claim 1, wherein melatonin or dose-equivalent melatonin analogue or melatonin receptor agonist is administered to the infant in suckled breast milk in two or more feedings at different times, wherein no more than about 1 mg of melatonin or dose-equivalent melatonin analogue or melatonin receptor agonist is administered at a single feeding.

18. The method of claim 17, wherein the infant's ingestion times of melatonin or dose-equivalent melatonin analogue or melatonin receptor agonist are separated by no more than about 14 hours.

19. A method for achieving a chronobiologic effect on a circadian rhythm in an infant to accommodate its mother or other caregiver, the method comprising the step of administering directly to the infant a concentration of melatonin or dose-equivalent melatonin analogue or melatonin receptor agonist sufficient to have a chronobiologic effect provided that no more than 1 mg of melatonin or dose-equivalent melatonin analogue or melatonin receptor agonist is directly administered to the infant at a single feeding, wherein a chronobiologic effect on the infant is achieved.

20. A method according to claim 19, wherein melatonin or dose-equivalent melatonin analogue or melatonin receptor agonist is administered directly to the infant in formula or expressed breast milk.

21. A method according to claim 20 wherein an amount of melatonin or dose-equivalent melatonin analogue or melatonin receptor agonist is added to said expressed breast milk.

22. A method according to claim 20 wherein said expressed breast milk is banked breast milk to which an amount of melatonin or dose-equivalent melatonin analogue or melatonin receptor agonist is added.

23. A method according to claim 20, wherein melatonin or dose-equivalent melatonin analogue or melatonin receptor agonist is administered directly to the infant in formula to which an amount of melatonin or dose-equivalent melatonin analogue or melatonin receptor agonist is added.

24. A method according to claim 19, wherein melatonin or dose-equivalent melatonin analogue or melatonin receptor agonist is administered directly to the infant orally/sublingually, rectally, intravenously or transdermally.

25. A method according to claim 19 wherein the infant is a premature infant.

26. A method according to claim 19, wherein the melatonin or dose-equivalent melatonin analogue or melatonin receptor agonist is administered to the infant in an immediate, sustained, or delayed release formulation, or effective combinations thereof.

27. A method for achieving a chronobiologic effect on a circadian rhythm in a human fetus in utero, the method comprising the step of having a pregnant woman bearing the fetus receive an amount of melatonin or dose-equivalent melatonin precursor, melatonin analogue or melatonin receptor agonist to produce a concentration of melatonin or dose-equivalent melatonin analogue or melatonin receptor agonist sufficient to have a chronobiologic effect on the fetus, provided that no more than about 1 mg melatonin or dose-equivalent melatonin analogue or melatonin receptor agonist is administered to the fetus within a 24-hour period, wherein a chronobiologic effect on the infant is achieved thereby.

28. The method of claim 26 wherein the circadian rhythm is the sleep/wake cycle circadian rhythm.

29. The method of claim 26 wherein the pregnant woman receives an amount of melatonin or dose-equivalent melatonin precursor, melatonin analogue or melatonin receptor agonist sufficient to have a chronobiologic effect on the fetus, provided that the first dose of melatonin or dose-equivalent melatonin precursor, melatonin analogue or melatonin receptor agonist is administered to the pregnant woman at a time that is about 14 hours after the pregnant woman's wake-up time.

30. The method of claim 26 wherein the melatonin or dose-equivalent melatonin precursor, melatonin analogue or melatonin receptor agonist is administered to the pregnant woman in an immediate, sustained, or delayed release formulation, or effective combinations thereof.

31. The method of claim 26, wherein the pregnant woman receives melatonin or dose-equivalent melatonin precursor, melatonin analogue or melatonin receptor agonist in a two or more doses at different times, wherein the dose is administered within a time interval of less than about 14 hours.

31. A method according to claim 26, wherein melatonin or dose-equivalent melatonin precursor, melatonin analogue or melatonin receptor agonist is administered to the pregnant woman orally, sublingually, rectally or transdermally.
